(19) United States
(12) Patent Application Publication
(10) Pub. No.: US 2019/0059555 A1
(43) Pub. Date: Feb. 28, 2019

(54) METHOD AND APPARATUS FOR SAMPLING COSMETICS AND PROCESS FOR USING AND PRODUCING SAME

(71) Applicants: Alexandra Hogg, Bella Vista, AR (US); Megan Roberson, Longmont, CO (US)

(72) Inventors: Alexandra Hogg, Bella Vista, AR (US); Megan Roberson, Longmont, CO (US)

(21) Appl. No.: 15/826,386

(22) Filed: Nov. 29, 2017

Related U.S. Application Data
(60) Provisional application No. 62/552,006, filed on Aug. 30, 2017.

(51) Int. Cl. A45D 40/00; A45D 40/30; G01J 3/02; G01J 3/52; B65D 75/32; B65D 75/58

(52) U.S. Cl. CPC A45D 40/0087; A45D 40/30; G01J 3/0267; A61K 2800/87; B65D 75/326; B65D 75/5855; A45D 2200/052; G01J 3/526

(57) ABSTRACT

A method and apparatus for sampling cosmetics and process for producing same comprising a fan deck being a fastened collection of cards, each being a clear plastic substrate on which an accurate color reproduction of a cosmetics product and associated identifying information has been printed.

### CROSS REFERENCE TO RELATED APPLICATION

This application claims priority to the following provisional application: U.S. Ser. No. 62/552,006 filed Aug. 30, 2017 entitled: METHOD AND APPARATUS FOR SAMPLING COSMETICS AND PROCESS FOR PRODUCING SAME, the contents of which are incorporated herein by reference.

### TECHNICAL FIELD

The present invention relates to cosmetics, cosmetics application, color-matching, and printing.

### BACKGROUND OF THE INVENTION

Cosmetics are substances used to enhance or alter the appearance of the face or body. They are generally mixtures of chemical compounds, some being derived from natural sources and others being synthetic. Cosmetics applied to the face to enhance its appearance are often called make-up or makeup. Makeup refers primarily to products containing color pigments that are intended to alter the user's appearance. Cosmetics that are meant to be used on the face and eye area are usually applied with a brush, a makeup sponge, or the fingertips or via a stick or bar. Common cosmetics include lipstick, eye shadow, foundation, and rouge, among many others.

Eye shadow is a cosmetic that is applied on the eyelids and under the eyebrows. Eye shadow comes in many different colors and textures. It is usually made from a powder and mica, but can also be found in liquid, pencil, cream or mousse form. Foundation is a skin-colored makeup applied to the face in order to even out complexion color, cover flaws, and/or change the natural skin tone. Rouge, also called blush or blusher, is a cosmetic typically used to redden the cheeks so as to provide a more youthful appearance. Modern rouge generally consists of a red-colored talcum-based power that is applied with a brush to the cheek.

Lipsticks are intended to add color and texture to the lips and come in a wide range of colors. Lipsticks may additionally impart properties other than color such as matte, satin, and luster, sometimes referred to as “finishes.”

Spectrophotometry is the study or practice of quantitative measurement of the reflective or transmissive properties with respect to light of a material as a function of the wavelength of said light. It is more specific than the general term electromagnetic spectroscopy in that spectrophotometry deals with visible light, near-ultraviolet, and near-infrared. According to Wikipedia, a photometer, generally, is an instrument that measures light intensity or the optical properties of solutions or surfaces. Photometers can measure illuminance, irradiance, light absorption, light scattering, light reflection, fluorescence, phosphorescence and/or luminescence. Most photometers detect the light with photoresistors, photodiodes or photomultipliers. To analyze the light, the photometer may measure the light after it has passed through a filter or through a monochromator for determination at defined wavelengths or for analysis of the spectral distribution of the light.

Spectrophotometry uses photometers, known as spectrophotometers, that measure a light beam's intensity as a function of its wavelength. A spectrophotometer is commonly used for the measurement of transmittance or reflectance of solutions, transparent or opaque solids, such as polished glass, or gases. The absorption of light is due to the interaction of light with the electronic and vibrational modes of molecules. Each type of molecule has an individual set of energy levels associated with the structure of its chemical bonds and nuclei, and will therefore absorb light of specific wavelengths. A molecule therefore has unique spectral properties dependent upon its specific and distinct chemical and atomic structure.

The use of spectrophotometers spans various scientific fields, such as physics, materials science, chemistry, biochemistry, and molecular biology. They are widely used in many industries including semiconductors, laser and optical manufacturing, printing and forensic examination, as well in laboratories for the study of chemical substances.

In printing, a drawdown or pulldown is created by manually applying a sample of production ink to a sample of production substrate, by pressing, hand-rolling, or by machine, in order to determine or evaluate accuracy of color matching between actual ink on substrate and another representation, often a digital image or stored ink sample.

Currently, individuals wishing to sample cosmetics must do so in person at a cosmetics counter in a department store or similar location, or by physically acquiring and applying, or having applied, to themselves the product to be sampled. This is inconvenient as it requires actual application of each sampled cosmetics product, making it a significant time commitment and limiting the number of cosmetics sampled to those physically available.

There exist temporary clear template cards meant for short-term, localized sharing or sampling of cosmetics products, requiring manual application of said cosmetics product thereon by a user, the quality of the resulting sample cards being thus dependent on steady-handedness even application by the user. Further, the temporary cards, having actual cosmetics product applied thereon, are prone to smudging and so not meant to be kept or stored. They require physical possession by a prospective user of the cosmetics product to be sampled and require significant work on the part of said user including preparation of card for cosmetics application, multiple applications and drying sessions of said cosmetics product to said card, and evaluation of result for color accuracy.

What is desired is an article allowing for fast, convenient, accurate sampling of multiple cosmetics that is consistent, re-usable and durable and which requires no cosmetics application nor actual cosmetics product. The present invention provides such an article, a method of using and process of making same.

### SUMMARY OF THE INVENTION

The invention is a method and apparatus for sampling cosmetics and process for producing same comprising a fan deck being a fastened collection of cards, each being a clear plastic substrate on which an accurate color reproduction of a cosmetics product and associated identifying information has been printed. A first embodiment uses a screw coupling means to hold the cards in the fan deck. A second embodiment uses a ring coupling means to hold the cards in the fan deck. In a first aspect, a substantially life-size lip design is used with each card in the deck having a different lip stick or lip color and identifying information. In a second aspect, each card in the deck has a different skin tone color. A deck can have a mix of lip designs and skin tone colors. To those skilled in the art to which this invention relates, many changes in construction and widely differing embodiments and applications of the invention will suggest themselves without departing from the scope of the invention as defined herein. The disclosures and the descriptions herein are purely illustrative and are not intended to be in any sense limiting.

### BRIEF DESCRIPTION OF THE DRAWINGS

For a better understanding of the present invention including the features, advantages and embodiments, reference is made to the following detailed description along with accompanying Figures, wherein:

FIG. 1 is an illustration of a perspective view of first aspect (lip design) of a first embodiment (screw coupling means) of the invention with a single card fanned out;

FIG. 2 is an illustration of a top plan view of first aspect (lip design) a first embodiment (screw coupling means) of the invention showing a plurality of the cards fanned out;

FIG. 3 is an illustration of a first aspect (lip design) of a first embodiment (screw coupling means) of the invention showing the invention in use;

FIG. 4 is an illustration of a perspective view of second aspect (skin tone swatch) of a first embodiment (screw coupling means) of the invention with a single card fanned out;

FIG. 5 is an illustration of a top plan view of second aspect (skin tone swatch) of a first embodiment (screw coupling means) of the invention showing a plurality of the cards fanned (color figure);

FIG. 6 is an illustration of a perspective view of first aspect (lip design) of a second embodiment (ring coupling means) of the invention;

FIG. 7 is an illustration of a perspective view of first aspect (lip design) of a third embodiment (nostril cut-out) of the invention with a single card fanned out;

FIG. 8 is a side view of a first embodiment (screw coupling means) of the invention;

FIG. 9 is a side view of a second embodiment (ring coupling means) of the invention; and

FIGS. 10A and 10B is a flow chart of the process of making the invention.

### DETAILED DESCRIPTION

While the making and using of the disclosed embodiments of the present invention is discussed in detail below, it should be appreciated that the present invention provides many applicable inventive concepts which can be embodied in a wide variety of contexts. Some features of the preferred embodiments shown and discussed may be simplified or exaggerated for illustrating the principles of the invention.

The method, apparatus, and process described herein make use of lipstick as an example of a cosmetics product to be sampled but one skilled in the art would recognize that lipstick is interchangeable in said method, apparatus, and process with eye shadow, foundation, rouge, and other cosmetics.

In one embodiment of the present invention, the apparatus is a fan deck comprising an assortment of printed cosmetics sample cards, along with a front and back cover, fastened together at a common point by a releasable fastening means being a screw, pin, ring, or other suitable releasable fastening means. Cards belonging to the same fan deck may share a common manufacturer or manufacturer line, or a common color or other characteristic, or may have no commonalities. In one embodiment said common fastening point is a hole punched or drilled through each card in a deck at the same relative location, forming an open channel running from the first through the last card of the deck when said deck is assembled by stacking of associated cards, said open channel admitting said releasable fastening means such that said cards are not removable from said deck but are able to pivot independently from each other about the longitudinal axis of said open channel when said fastening means is engaged. When said fastening means is released, cards may be removed from and/or added to said deck.

Each card consists of a clear plastic substrate having certain dimensions, defining an attached end as being the closest end to said common attachment point and a free end as being opposite said attached end, and on which a reproduction of an applied cosmetics product is printed, said reproduction being accurate as to color and form and comprising a deposited layer of primer and one or more subsequently deposited layers of ink, said ink being formulated for accurate color matching to a master color sample of actual applied cosmetics product and improved printing properties including application to and retention by plastic substrate of said ink and durability of resulting print including resistance to moisture and heat. Each card further comprises a strip located at the free end of said card comprising a layer of primer and one or more subsequent layers of said formulated ink, such that said card's associated cosmetics product sample color is visible when said free end is viewed edge-on. Each card additionally comprises a common fastening point, being in one embodiment a hole punched or drilled through each card in a deck at the same relative location, forming an open channel running from the first through the last card of the deck when said deck is assembled by stacking of associated cards. Additional Figures disclose the use of the invention as a foundation card and with other cosmetics.

In a further embodiment of the present invention, a user uses said apparatus to sample cosmetics by first acquiring a fan deck then either flipping through said deck or viewing said deck edge-on and identifying a desired color sample. Said user then fans out said deck, locates the card containing said desired sample and separates it from the remaining cards in said deck by rotating said remaining cards about the fastening means and out of the way. Said user then holds the unobstructed separated card up to a facial feature of interest, and, using a mirror, compares or evaluates the printed color sample against said facial feature, said feature being visible through the clear substrate of said card. Said user can then make note of the color name and/or other information present on the card if desired.

In a further embodiment of the invention, a cosmetics sample fan deck is created by a process comprising the steps of: preparing a master color sample; measuring certain material properties of said master color sample using a color spectrophotometer; formulating an ink sample using said measurements; creating a color drawdown by applying said formulated ink sample to a card having properties identical to those of a production card, namely being a clear plastic substrate on which a primer has been deposited; evaluating said drawdown for closeness of match of color and finish between said drawdown and the master color sample, a digital image, and/or other representation; once a satisfactory ink formulation has been found, creating a small production batch of said ink formulation, to which chemical elements are added which aid in printing; running a test print of satisfactory ink formulation on production card blanks which have been prepared by printing primer on to said blanks in a prescribed shape, in this case, lips, which has been digitally created and a press plate made thereof; printing via printing press a final ink formulation in a prescribed shape on to sheets of card blanks, each of which has been prepared with printed primer in said prescribed shape; printing an associated cosmetics product name on each card; collating printed sheets, stacking same in a desired order; cutting cards of desired dimensions out of said sheets using a hydraulic die cutter; drilling through each stack of cards a common attachment point (small hole); collating and ordering cards required for a given fan deck; adding to the front and back of said deck pre-printed front and back covers, respectively; inserting fastening means through deck common attachment point; and securing fastening means.

The steps of master color sample comprise the steps of depositing a primer in a prescribed shape on a card comprising a clear plastic substrate and then manually applying to said card, over said primer, the actual cosmetics product to be matched, making multiple applications as needed to most closely replicate on said card the color and finish of said cosmetic as applied to a facial area of interest. Once a satisfactory likeness is achieved, said card containing said actual cosmetic is considered a master color sample.

Referring now to the drawings, FIG. 1 is an illustration of a perspective view of first aspect (lip design) of a first embodiment (screw coupling means) of the invention with a single card fanned out. As seen therein a fan deck 100 comprises an assortment of printed cosmetics sample cards 101, along with a front cover 102 and back cover 103, fastened together at a common point 104 by a permanent or releasable fastening means 105. A plurality of cards 101 belonging to the same fan deck may share a common manufacturer or manufacturer line, or a common color or other characteristic, or may have no commonalities. The name of the color on the card 101 is imprinted along a portion of a side 106 of the card 101. In one embodiment said common fastening point 104 is a hole punched or drilled through each card in a deck at the same relative location, forming an open channel running from the first through the last card of the deck when said deck is assembled by stacking of associated cards 101, said open channel admitting said releasable fastening means 105 such that said cards 101 are not removable from said fan deck 100 but are able to pivot independently from each other about the longitudinal axis of said open channel when said fastening means 105 is engaged. When said fastening means 105 is released, cards 101 may be removed from and/or added to said deck. As seen in FIG. 1, the fastening means 105 comprises a sleeve nut with mating bolt. Other possible fastening means include a barrel bolt and mated bolt, Chicago screw and mated bolt, post and screw, pin, connector bolt and threaded barrel nut or other suitable coupling means.

Each card 101 consists of a clear plastic substrate 107 having a generally rectangular shape having a vertical height (when placed against a user's face) of between 1.5 and 3 inches and a horizontal length of between 2 and 4 inches. Proximate a corner of each card 101 of the fan deck 100 is attached end being the common attachment point and a free end opposite said attached end. On each card 101 is printed a reproduction of an applied cosmetics product 108, said reproduction being accurate as to color and form and comprising a deposited layer of primer and one or more subsequently deposited layers of ink, said ink being formulated for accurate color matching to a master color sample of actual applied cosmetics product. Improved printing properties including application to and retention by plastic substrate 107 of said ink and durability of resulting print including resistance to moisture and heat.

As seen in FIG. 1, the applied cosmetics product 108 is dimensioned in the outline of human lips 108 and is colored with the color representing a lipstick color or lip color. Such color includes any color in a shade selected from the group of red, pink, purple, orange, yellow, green, blue, peach, brown, black, gray and flesh-tone. Each card 101 further comprises a strip 106 located along the free end of said card 101 with a printed name of the color, the strip and printing comprising a layer of primer and one or more subsequent layers of said formulated ink. The orientation of the printing of the card's 101 associated cosmetics product sample color name is visible when said free end of card 101 is viewed edge-on. Each card 101 additionally comprises a common fastening point, being in one embodiment a hole punched or drilled through each card 101 in the fan deck 100 at the same relative location, forming an open channel running from the first through the last card 101 of the fan deck 100 when said fan deck 100 is assembled by stacking of associated cards 101. Additional Figures disclose the use of the invention as a foundation card having skin tone swatches. Additional aspects of the invention include, but are not limited having imprinted on the plastic substrate 107 of cards 101 foundations swatches, blush swatches and eye shadow swatches. With respect to foundation and skin swatches, the color thereon can be any color or shade found on the Von Luschan's chromatic scale.

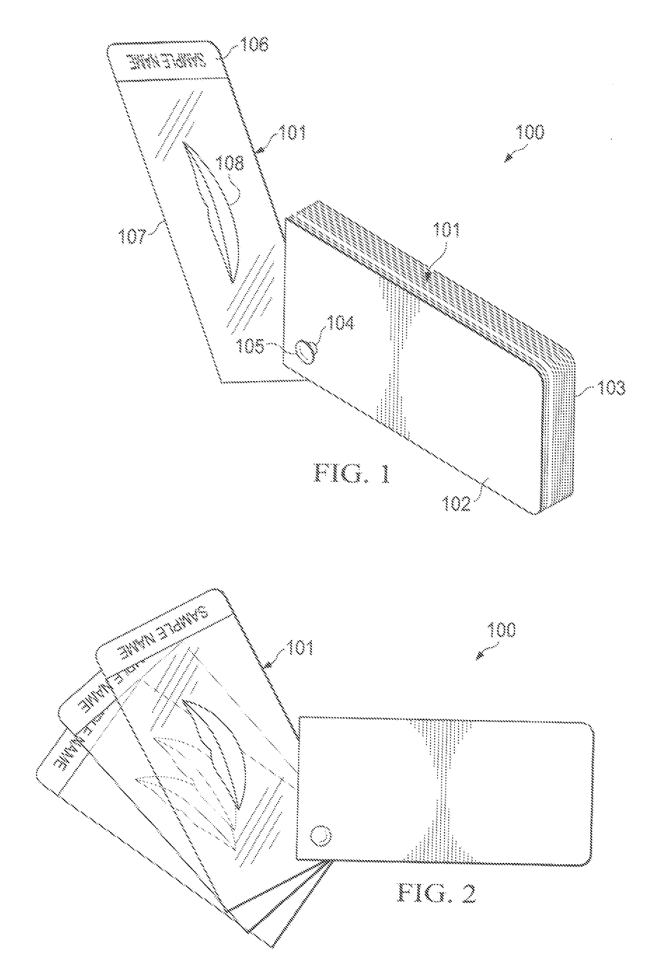

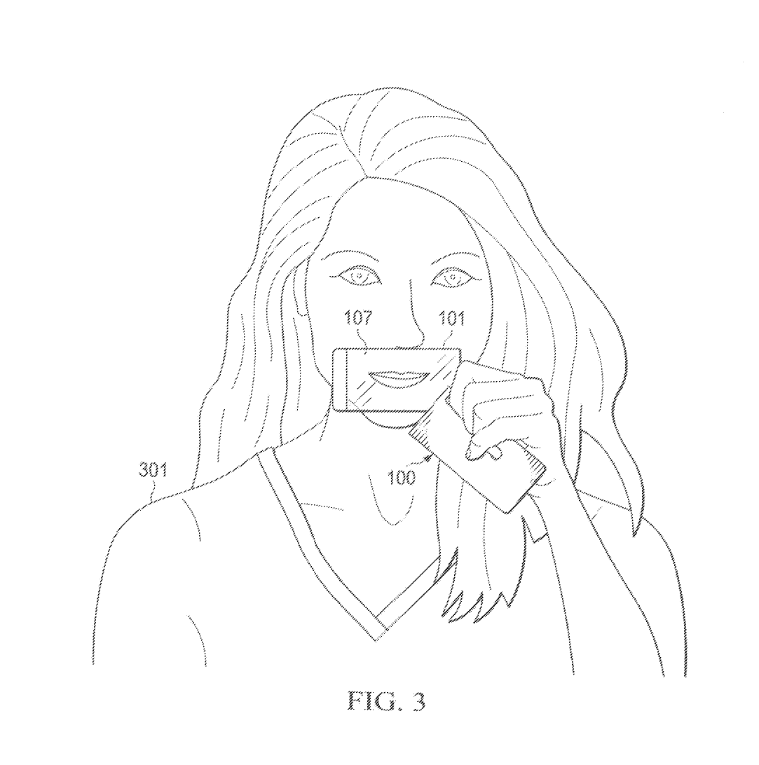

FIG. 2 is an illustration of a top plan view of first aspect (lip design) a first embodiment (screw coupling means) of the invention showing a plurality of the cards 101 fanned out.

FIG. 3 is an illustration of a first aspect (lip design) of a first embodiment (screw coupling means) of the invention showing the invention in use. As seen therein, a user 301 uses the fan deck 100 to sample cosmetics by first acquiring a fan deck 100 then either flipping through the cards 101 of said fan deck 100 or viewing said fan deck 100 edge-on and identifying a desired color sample by reference to information printed on the strip 106. Said user 301 then fans out said fan deck 100, locates the card 101 containing said desired sample and separates it from the remaining cards 101 in said fan deck 100 by rotating said remaining cards 101 about the fastening means 105 and out of the way. Said user then holds the unobstructed separated card 101 up to a facial feature of interest, and, using a mirror, compares or evaluates the printed color sample against said facial feature, said feature being visible through the clear substrate 107 of said card 101. Said user can then make note of the color name and/or other information present on the card 101 if desired.

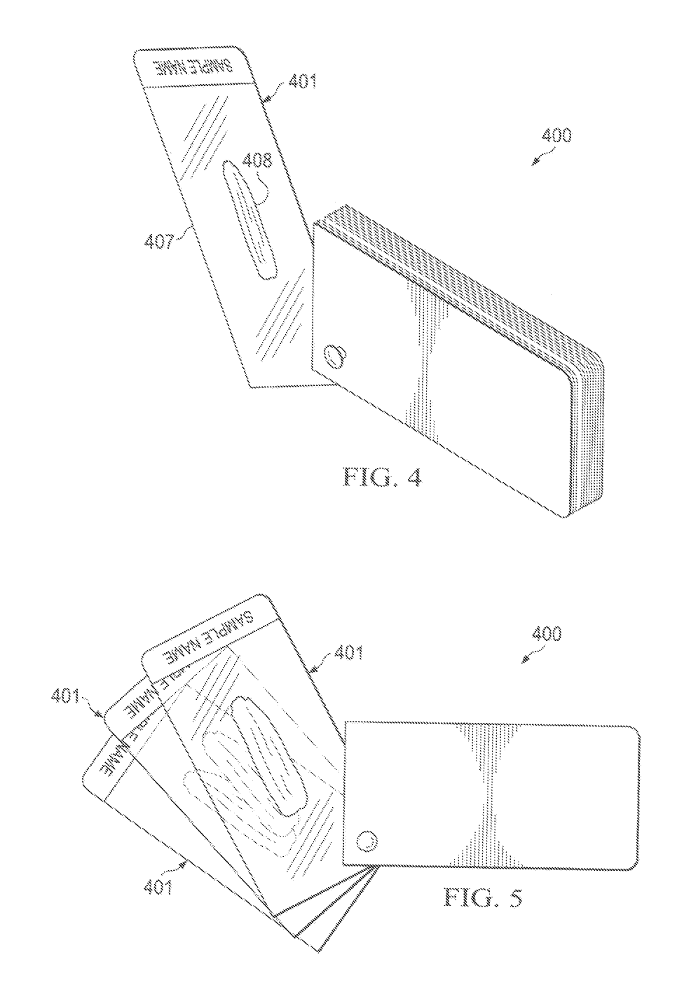

FIG. 4 is an illustration of a perspective view of second aspect (skin tone swatch) of a first embodiment (screw coupling means) of the fan deck 400 with a single card 401 fanned out. As seen therein, card 401 consists of a clear plastic substrate 407 having certain dimensions, defining an attached end as being the closest end to said common attachment point and a free end as being opposite said attached end. The reproduction of an applied cosmetics product 408 is printed on card 401. As seen in FIG. 4, the applied cosmetics product 408 is dimension in the outline of a swatch and the color comprises a human skin tone.

FIG. 5 is an illustration of a top plan view of second aspect (skin tone swatch) of a first embodiment (screw coupling means) of the fan deck 400 showing a plurality of the cards 401 fanned (color figure).

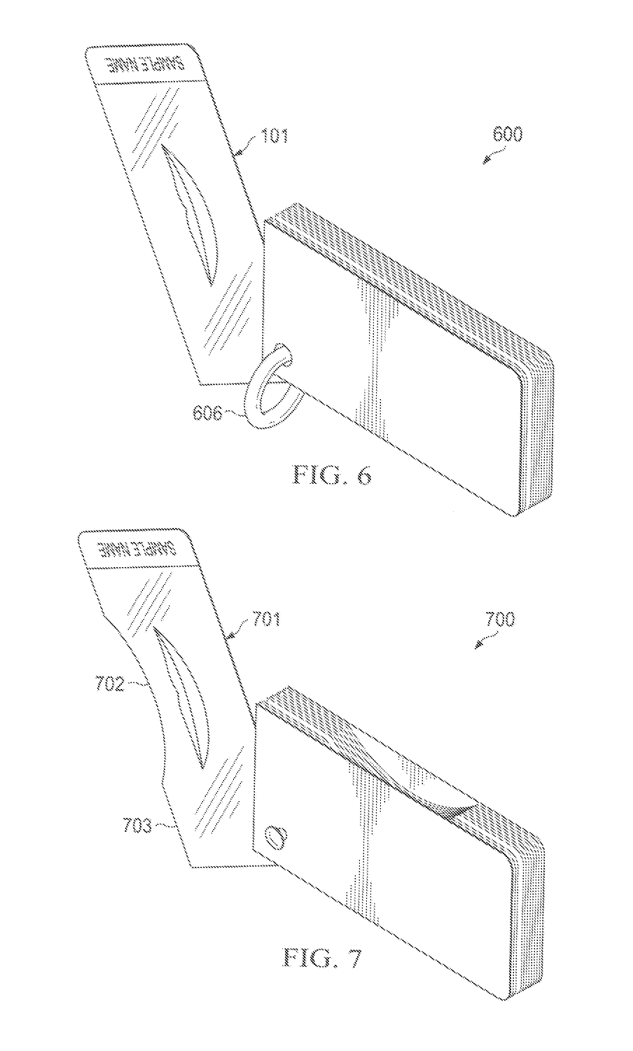

FIG. 6 is an illustration of a perspective view of first aspect (lip design) of a second embodiment (ring coupling means) of the fan deck 600 with cards 101. As seen therein, fastening means 606 comprises a ring connector. Alternative fastening means further include releasable spring snaps in circular or oval shapes.

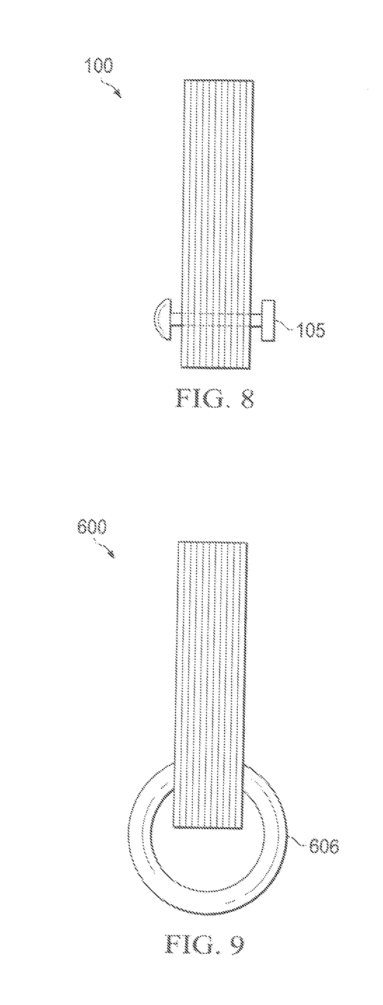

FIG. 7 is an illustration of a perspective view of first aspect (lip design) of a third embodiment (nostril cut-out) of the fan deck 700 with a single card 701 fanned out. As seen therein each card 701 has a curved cut-out 702 along the top edge 703 of the card 701 allowing a space for the lower nostril of the user. The curved carve-out has an arc angle of between ten and fifty degrees and an arc length of between about seven to fifteen times an arc height. The arc length, L, of an arc of a circle with radius r and subtending an angle θ (measured in radians) with the circle center, i.e., the central angle, equals θ*r. A practical way to determine the length of an arc in a circle is to plot two lines from the arc's endpoints to the center of the circle, measure the angle where the two lines meet the center, then solve for L by cross-multiplying the statement: measure of angle in degrees/360°=L/circumference.

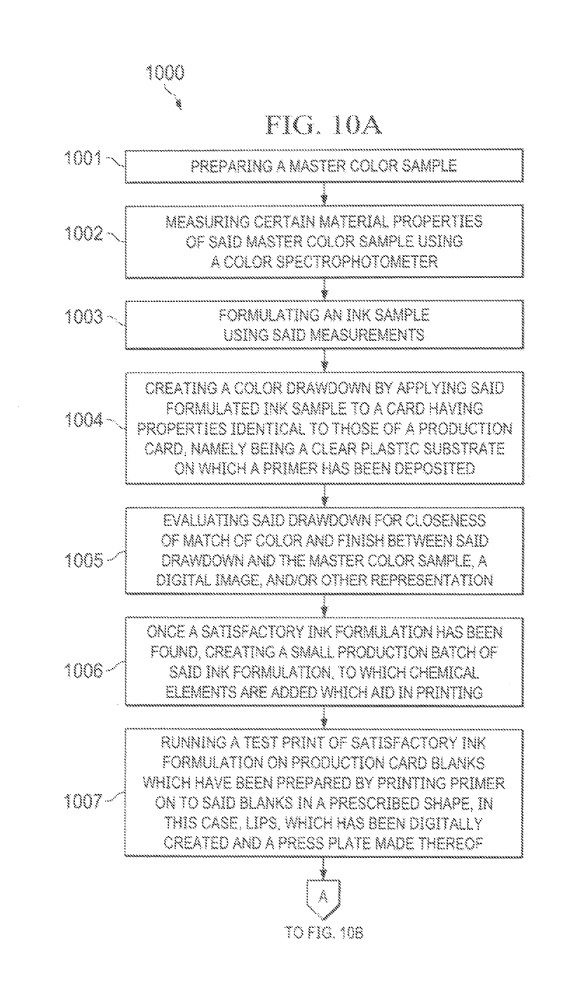

FIG. 8 is a side view of a first embodiment (screw coupling means) of the fan deck 100 and FIG. 9 is a side view of a second embodiment (ring coupling means) of the fan deck 600.

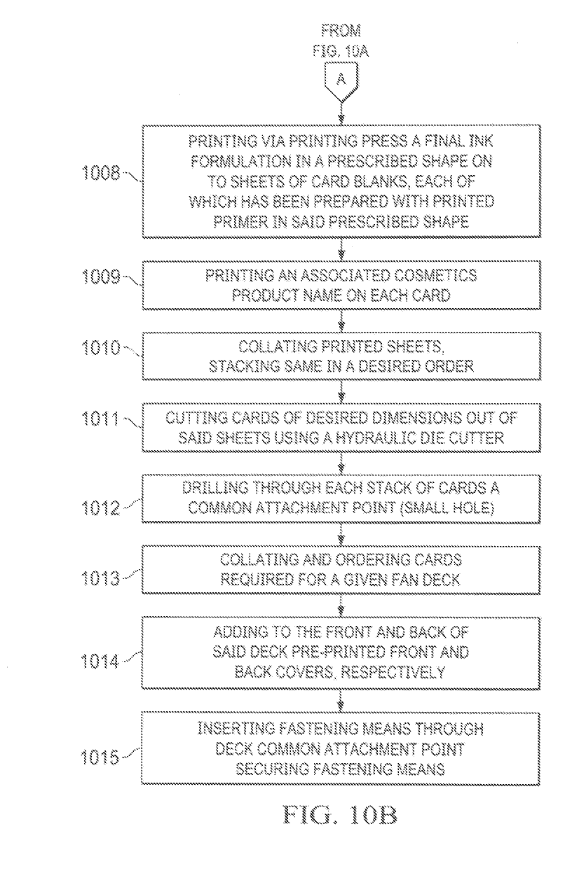

FIGS. 10A and 10B is a flow chart of the process 1000 of making the invention. As seen therein, in step 1001, a master color sample is prepared. In step 1002, certain material properties of said master color sample are measured using a color spectrophotometer. In step 1003, an ink sample using said measurements is formulated. In step 1004, a color drawdown is created by applying said formulated ink sample to a card having properties identical to those of a production card, namely being a clear plastic substrate on which a primer has been deposited. In step 1005, the drawdown is evaluated for closeness of match of color and finish between said drawdown and the master color sample, a digital image, and/or other representation. In step 1006, once a satisfactory ink formulation has been found, a small production batch of said ink formulation is created, to which chemical elements are added which aid in printing. In step 1007, a test print of satisfactory ink formulation is run on production card blanks which have been prepared by printing primer on to said blanks in a prescribed shape, in an aspect of an embodiment, lips, which has been digitally created and a press plate made thereof. In step 1008, a final ink formulation is printed via a printing press in a prescribed shape on to sheets of card blanks, each of which has been prepared with printed primer in said prescribed shape. In step 1009, an associated cosmetics product name is printed on each card. In step 1010, printed sheets are collated, stacking same in a desired order. In step 1011, the cards are cut of desired dimensions out of said sheets using a hydraulic die cutter. In step 1012, each stack of cards is drilled at a common attachment point (small hole). In step 1013, the cards are collated and ordered as required for a given fan deck. In step 1014, pre-printed front and back covers are added to the front and back of the deck. In step 1015, the fastening means is added through deck common attachment point and in step 1016, the fastening means is secured.

The embodiments shown and described above are only exemplary. Even though numerous characteristics and advantages of the present invention have been set forth in the foregoing description together with details of the circuits and methods of the present invention, the disclosure is illustrative only and changes may be made within the principles of the invention to the full extent indicated by the broad general meaning of the terms used herein. Various alterations, modifications and substitutions can be made to the circuits and methods of the disclosed invention and the fabrication system that implements the present invention without departing in any way from the spirit and scope of the invention.

What is claimed is:

1. An apparatus for facilitating the sampling cosmetics comprising:
a fan deck comprising a plurality of stacked cosmetics product sample cards; and
a fastening means for fastening an end of the stacked cosmetics product sample cards at a common attachment point.

2. The apparatus of claim 1, wherein at least one of the cosmetics product sample cards comprises a clear plastic substrate having a generally rectangular shape.

3. The apparatus of claim 1, wherein at least one of the stacked cosmetics product sample cards includes thereon a reproduction of an applied cosmetics product accurate as to color and form.

4. The apparatus of claim 3, wherein the reproduction of an applied cosmetics product is dimensioned in the form of human lips.

5. The apparatus of claim 4, wherein each reproduction of an applied cosmetics product is colored in a shade selected from the group of red, pink, purple, orange, yellow, green, blue, peach, brown, black, gray and flesh-tone.

6. The apparatus of claim 3, wherein the reproduction of an applied cosmetics product is dimensioned in the form of a swatch; and
the applied cosmetics product is colored in a shade corresponding to one of a foundation swatch, blush swatch or eye shadow swatch.

7. The apparatus of claim 6, wherein the reproduction of an applied cosmetics product is dimensioned in the form of a swatch and the applied cosmetics product has a color identifiable on the Von Luschan's chromatic scale.

8. The apparatus of claim 3, wherein the reproduction of an applied cosmetics product comprises a layer of primer and one or more subsequent layers of formulated ink, said ink being formulated for accuracy of color match to actual applied cosmetics product.

9. The apparatus of claim 1, further comprising a printed strip running the full vertical height of at least one cosmetics product sample card at its free end, said printed strip comprising a layer of primer and one or more subsequent layers of ink, the printing identifying a color associated with the applied cosmetics product.

10. The apparatus of claim 1, wherein the common attachment point comprises a punched or drilled hole through the same relative location on each card.

11. The apparatus of claim 1, wherein the fastening means comprises a sleeve nut combined with a mating bolt.

12. The apparatus of claim 1, wherein the fastening means comprises one selected from the group of a barrel bolt and mated bolt, Chicago screw and mated bolt, post and screw, pin, and connector bolt and threaded barrel nut.

13. The apparatus of claim 1, wherein at least one of the cosmetics product sample cards comprises a clear plastic substrate having a generally rectangular shape with a curved carve-out on a top edge thereof.

14. The apparatus of claim 13, wherein the curved carve-out has an arc angle of between ten and fifty degrees and an arc length of between about seven to fifteen times an arc height.

15. A method for sampling cosmetics, comprises the steps of
identifying a desired sample color;
locating said color on a card from a plurality of cards held together at a common attachment point in a fan deck;
separating the card containing said color from rest of the fan deck;
holding said card up to facial feature of interest;
evaluating said sample color using a mirror; and
noting sample color name and/or other information printed on the card.

16. The method of claim 15, wherein the fan deck comprises a plurality of cosmetics product sample cards further comprising a clear plastic substrate having a generally rectangular shape wherein at least one of the stacked cosmetics product sample cards includes thereon a reproduction of an applied cosmetics product accurate as to color and form.

17. The method of claim 14, wherein the reproduction of an applied cosmetics product is in the form of lips and is colored in a shade selected from the group of red, pink, purple, orange, yellow, green, peach and flesh-tone.

18. The method of claim 15, wherein the fan deck comprises a plurality of cosmetics product sample cards further comprising a clear plastic substrate having a generally rectangular shape with a curved carve-out that has an arc angle of between ten and fifty degrees and an arc length of between about seven to fifteen times an arc height.

19. A method of creating a cosmetics article, comprising the steps of:
preparing a master color sample;
measuring certain material properties of said master color sample using a color spectrophotometer;
formulating an ink sample using said measurements;
creating a color drawdown by applying said formulated ink sample to a card having properties identical to those of a production card, namely being a clear plastic substrate on which a primer has been deposited;
evaluating said drawdown for closeness of match of color and finish between said drawdown and the master color sample, a digital image, and/or other representation;
once a satisfactory ink formulation has been found, creating a small production batch of said ink formulation, to which chemical elements are added which aid in printing;
running a test print of satisfactory ink formulation on production card blanks which have been prepared by printing primer on to said blanks in a prescribed shape, which has been digitally created and a press plate made thereof;
printing via printing press a final ink formulation in a prescribed shape on to sheets of card blanks, each of which has been prepared with printed primer in said prescribed shape;
printing an associated cosmetics product name on each card;
collating printed sheets, stacking same in a desired order;
cutting cards of desired dimensions out of said sheets using a hydraulic die cutter;
drilling through each stack of cards a common attachment point (small hole);
collating and ordering cards required for a given fan deck;
adding to the front and back of said deck pre-printed front and back covers, respectively;
inserting fastening means through deck common attachment point; and
securing fastening means.

20. The method of claim 19, wherein the prescribed shape is in the form of human lips and the ink formulation has a color selected from the group of red, pink, purple, orange, yellow, green, blue, peach, brown, black, gray and flesh-tone.
